(19) United States
(12) Patent Application Publication
(10) Pub. No.: US 2005/0251093 A1
(43) Pub. Date: Nov. 10, 2005

(54) Hand mounted surgical aspiration device

(76) Inventor: Hassan Abou-Kansoul, Stockholm (SE)

Correspondence Address: QUARLES & BRADY LLP, 411 E. WISCONSIN AVENUE, SUITE 2040, MILWAUKEE, WI, 53202-4497, US

(21) Appl. No.: 11/122,423

(22) Filed: May 5, 2005

Related U.S. Application Data
(60) Provisional application No. 60/568,868, filed on May 6, 2004.

(51) Int. Cl. A61M 1/00

(52) U.S. Cl. 604/119; 604/902; 206/570

(57) ABSTRACT

A finger clamp fitting over the proximal phalanx of a surgeon's finger holds a flexible aspiration tube near the tip of the surgeon's finger but away from the gripping plane so as to be easily bent, gripped and released over the course of surgery.

### CROSS-REFERENCE TO RELATED APPLICATIONS

This application claims the benefit of U.S. Provisional Application 60/568,868 filed May 6, 2004 hereby incorporated by reference.

### STATEMENT REGARDING FEDERALLY SPONSORED RESEARCH OR DEVELOPMENT

### BACKGROUND OF THE INVENTION

During surgery it is often necessary for the surgeon to aspirate blood and liquid from the wound or surgery site. Current surgical suction devices are held by the surgeon or an assistant and occupy the use of one hand. The person doing the suctioning loses the use of the hand holding the suction device. If an assistant is doing the suctioning, he or she may interfere with the surgeons view of the operative field or not place the suction device in the position needed or desired by the operating surgeon. If the person doing the suctioning needs to perform another function, the suction device must be handed off to another person or laid aside.

U.S. Pat. No. 5,120,305 to Sasaki describes a glove incorporating tubing dispensing sterile saline from the tips of the glove fingers and a palm aspirator for collecting those materials. This glove, when worn by the surgeon, may eliminate the need for a second person to operate the aspiration tube. Nevertheless, the glove is complex and would likely prove cumbersome particularly if the surgeon requires fine motor control over a needle or scalpel or the like. In this case, the glove would need to be removed, eliminating some of the benefits that it provides in convenience.

Sasaki indicates that the conduits for aspiration and scavenging may be detachable from the glove by means such as Velcro. Such Velcro attachments may still interfere with use of the surgeon's fingers when the conduits are detached. Further, the time required to attach and detach the conduits to the glove would likely provide a significant interruption to the surgical procedure beyond that required for the surgeon to simply locate and position a separate aspiration tube.

### BRIEF SUMMARY OF THE INVENTION

The present invention locates an aspiration tube at the surgeon's fingertips without interfering with alternative uses of the surgeon's fingers during surgery. Generally, in a principle embodiment, the invention attaches the aspiration tube to a single finger of the surgeon at a point removed from the fingertip. The tube then extends in cantilever toward the fingertips of the surgeon. By straightening then bending his or her finger, the surgeon may easily capture and direct the tube to the site to be aspirated or grasp the tube between the finger and thumb to direct it or control suction. Upon release, the tube flexes out of the way or may be retracted to allow free use of the surgeon's fingers. The mounting may be a simple ring fitting around the finger and easily molded or formed of a single plastic element.

Specifically then, the present invention provides a hand mounted aspiration device for surgery having an axially bendable aspiration tube providing an intake end receiving aspirated fluids. A clamp engages a finger of the surgeon and holds the flexible aspiration tube at a portion removed from the intake end to direct the intake end in cantilever fashion to a point grippable between a tip of the finger and thumb of the surgeon.

It is thus an object of the invention to provide an aspiration tube at the surgeon's fingertips, yet not connected to the surgeon's fingertips such as would interfere with alternative use of the surgeon's fingers.

The clamp holds the portion of the bendable aspiration tube so that a grasping of the intake end between the tip of the finger and the thumb allows for directing or bending of the aspiration tube. A release of the intake end from gripping moves the intake end away from the gripping point under its natural elasticity or allows it to be bent away.

It is thus another object of the invention to provide a mounting system that naturally holds the tube end away from the normal gripping point of the finger and thumb so as not to interfere with gripping of other elements such as needles or the like, and yet which may be easily captured by straightening then bending the finger and flexing the tube downward to be gripped.

The clamp may be a ring encircling the finger.

It is thus another object of the invention to provide a simple and universal method for holding the tube to the hand that works with a gloved hand and without the need for a specially prepared glove.

The ring may flex to expand in diameter.

It is thus another object of the invention to provide a clamping method that conforms to a variety of different finger sizes.

The clamp may hold the aspiration tube to allow sliding adjustment of a length of cantilever of the aspiration tube from the clamp to the intake end.

It is thus another object of the invention to allow individual adjustment of the length of the tube by the surgeon.

The clamp may hold the aspiration tube with a ring around the aspiration tube.

It is another object of the invention to provide a method of attaching the clamp to an aspiration tube without the need for adhesives or welds and which allows simple adjustment of the length of cantilever.

The ring around the tube may be sized to allow for adjustment and fitting of different size tubes.

It is thus another object of the invention to have a ring which allows the tube to fit securely and prevent accidental slippage of the tube within the ring yet allow for sliding of the tube both forward and back.

The clamp may be a pair of coplanar rings.

It is another object of the invention to align the axis of the aspiration tube with the axis of the surgeon's finger.

The device may include a wristband stabilizing an end of the aspiration tube opposite the intake end against a wrist of the surgeon.

It is thus another object of the invention to prevent the tubing attached to the aspiration tubing from pulling on the surgeon's finger or interfering during surgery.

The aspiration tube may terminate at an end opposite the intake end with a bayonet connector.

Thus it is another object of the invention to provide a lightweight disposable element that may be attached to a separate vacuum system.

The intake end may include axial openings and a radial end opening.

It is thus another object of the invention to allow manipulation of the tube end by the surgeon's fingers to control the aspiration flow either by covering and uncovering the side holes or squeezing the tube together.

The device may further include an aspiration flow valve controlling the flow of aspirated materials.

It is thus another object of the invention to provide a coarse flow control that may be used in addition to manipulation of the tube end by the surgeon.

The device may further include a vacuum line having a pre-formed helical configuration.

Another object of the invention is to provide a distal expandable telephone cord like cable which allows for unrestricted surgeon moving of the suction apparatus.

These particular objects and advantages may apply to only some embodiments falling within the claims and thus do not define the scope of the invention.

### BRIEF DESCRIPTION OF THE DRAWINGS

FIG. 1 is a perspective view of a sterile kit holding the elements of the present invention including a finger clamp, the aspiration tube, and a wristband;

FIG. 2 is a front and side view of the fingertip end of the aspiration tube and clamp of the present invention showing formation of the clamp as a pair of rings in a single continuous double spiral of a plastic strip and showing the cantilever length provided by the kit such as approximates the distance between the fingertip and the finger clamp;

FIG. 3 is a side elevational view of a surgeon's hand when wearing the aspiration tube of the present invention showing the correct adjustment of the cantilever length and a positioning of the tube in an unflexed state beneath a fingertip of the surgeon when the finger is fully extended;

FIG. 4 is a figure similar to that of FIG. 3 showing the flexing downward of the aspiration tube to be grasped between the finger and thumb and showing its upward flexure away from the point of contact of the index finger and thumb for alternative uses of the surgeon's hands;

FIG. 5 is a perspective view of the surgeon's hand and forearm showing use of the stabilizing Velcro band as a strain relief for the tubing and the attachment of a bayonet connector of the aspiration tube to a valve allowing coarse control of aspiration flow and an expandable telephone like cable allowing for stretching and moving of the suction device in the surgeon's hand;

FIG. 6 is a vertical, cross-sectional, side view of an alternative embodiment of the present invention using a finger cot fitting over a gloved finger to hold the tip of the aspiration tube at the fingertip; and

FIG. 7 is a vertical, cross-sectional, end view of the embodiment of FIG. 6 showing the groove in the finger cot releasably gripping the aspiration tube.

### DETAILED DESCRIPTION OF THE PREFERRED EMBODIMENT

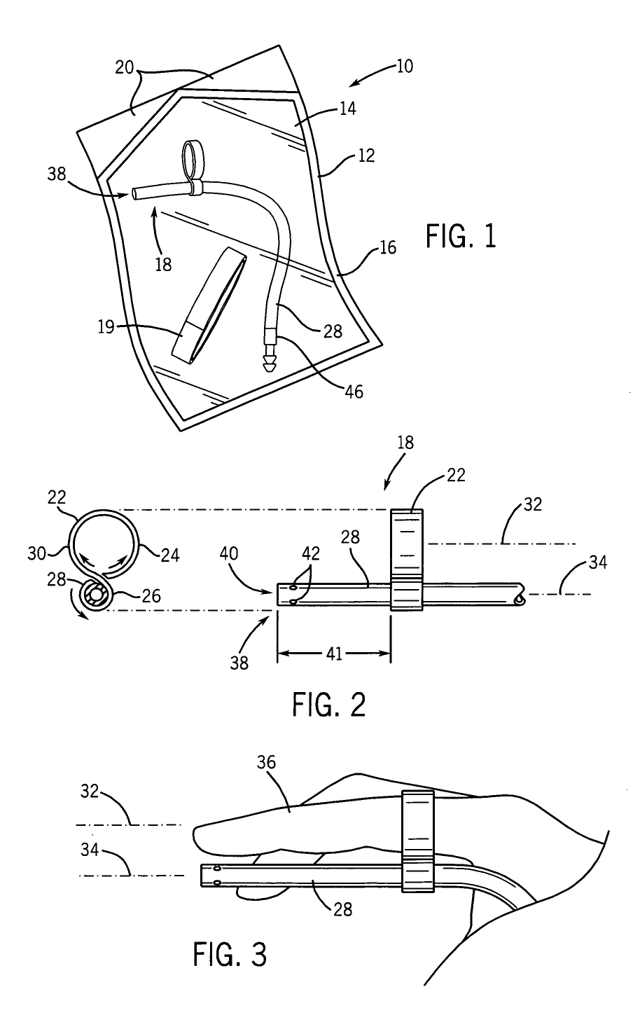

Referring now to FIG. 1, the present invention may provide a kit 10 including a disposable paper and plastic pouch 12 having a clear front panel 14 joined to a plastic coated paper panel by edge seal 16 to contain an aspiration tube assembly 18 and a wristband 19 in a sterile state within the pouch 12. Unsealed wings 20 of the pouch 12 allow the paper and plastic halves of the pouch 12 to be separated in the surgical environment to release the necessary kit elements.

Referring also to FIG. 2, the aspiration tube assembly 18 includes a tube clamp 22 providing a first ring 24 sized to receive a surgeon's finger at the base of the finger. The first ring 24 is attached to a smaller second ring 26 having a diameter sized to fit about the aspiration tube 28.

The first ring 24 and second ring 26 may be manufactured of a continuous strip 30 formed in a counterclockwise spiral to define the first ring 24 and, then in a clockwise spiral, to define the second ring 26 or vice versa for rings 24 and 26. The ends of the strip 30 remain free so as to allow an expansion of the diameter of the rings 24 and 26 by outward flexing of the strip 30. The size of the rings 24 and 26 without flexure are made less than their expected necessary diameters during use so as to provide a slight gripping for a received finger or tube 28. The formation of the rings 24 and 26 are such that an axis 32 of ring 24 is generally parallel to an axis 34 of ring 26.

The aspiration tube 28 may be, for example, a 3-7 mm internal diameter biocompatible silicone or other plastic. The aspiration tube 28 has an intake end 38 that will receive aspirated liquids and that include an axial opening 40 and one or more radial openings 42. The axial opening 40 is optional. The radial openings 42 may be several millimeters from the intake end 38 or may be located as far back as several centimeters from the intake end 38. Multiple rings of holes may be used distributed along the end of the aspiration tube.

Referring to FIGS. 1 and 3 in the kit 10, the clamp 22 is initially positioned on the aspiration tube 28 so that a distance 41 between the intake end 38 and the clamp 22 approximates the distance between the middle of the proximal phalanx bone of the finger 36 and the fingertip of the finger 36 for an average adult. Alternatively, the ring 24 may be worn about the middle phalanx bone and the distance 41 adjusted appropriately. The ring 24 further can be rotated so that the aspiration tube 28 is above or to the side of the finger 36, and depending on the preference of the surgeon, the position of the ring 24 and the distance 41 may be altered during surgery without the need to remove a glove or the like.

Referring now to FIGS. 2 and 3 when the ring 24 is placed on a finger 36 of the surgeon (for example, but not necessarily, the pointer finger) at the base of the finger, the axis of the tube 28 (identical to the axis 34 of the ring 26) is generally parallel to the surgeon's finger 36 when that finger 36 is extended to be straight. The tube 28 is elastic to naturally spring to a straight configuration over short lengths of several inches or is malleable to allow for bending. In this configuration, the aspiration tube 28 will extend along axis 34 parallel to and beneath the finger 36.

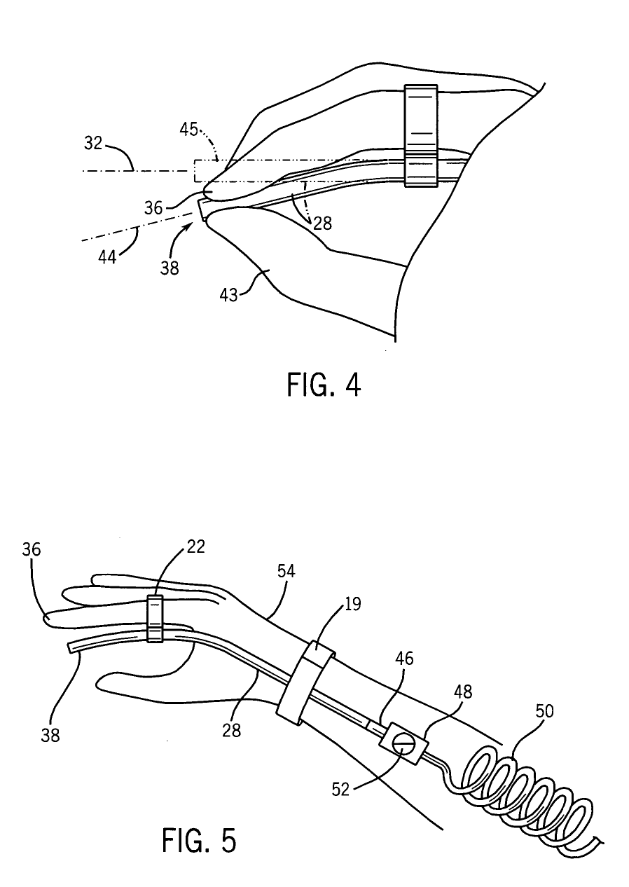

Referring now to FIG. 4, the tube 28 may be gripped between the fingertip of pointer finger 36 and the thumb 43 at a gripping plane 44 by downward flexure of the tube 28 away from axis 32 defined by the orientation of the proximal phalanx of the finger 36. In this configuration, the flow and location of the intake end 38 may be carefully controlled by the surgeon's finger. Release of the tube 28 allows it to spring upward as indicated by dotted lines 45 away from the plane 44 to allow the surgeon's fingers to tie knots, or grip alternative instruments or tissue without interference from the tube 28. When the tube 28 is required again, straightening of the surgeon's fingers per FIG. 3 allows it to be captured again and moved down through flexure to the gripping plane 44.

Referring now to FIG. 5 and to FIG. 1, an end of the tube 28 removed from the intake end 38 may include a bayonet plug 46 that may be received by a valve socket 48 providing for a connection to an auxiliary vacuum line 50 and for control by means of a valve control knob 52 of the coarse flow through the tube 28. The valve of the valve socket 48 may be any of a number of different types including those providing an adjustable door or sleeve opening over the tube to admit ambient air for those having an adjustable internal air shutter, or an adjustable IV line roller or ratchet-type adjustment clamp well known in the art.

The Velcro wristband 19 is a strip of material having hooks on one side and loops on the other side to allow it to be wrapped and attached about the wrist. The Velcro wristband 19 may be wrapped about the wrist of the surgeon over the glove 54 to absorb the strain caused by the weight of the vacuum line 50 at the wrist without pulling on the finger 36 of the surgeon. The auxiliary vacuum line 50 may have a coil structure, like a telephone cord, to prevent kinking and excess slack.

In an alternative embodiment, the tube 28 may be a stiffer material having an accordion pleat or incorporated wire or be malleable that allows it to be flexed or bent out of the way and thus to be effectively resilient without being elastic.

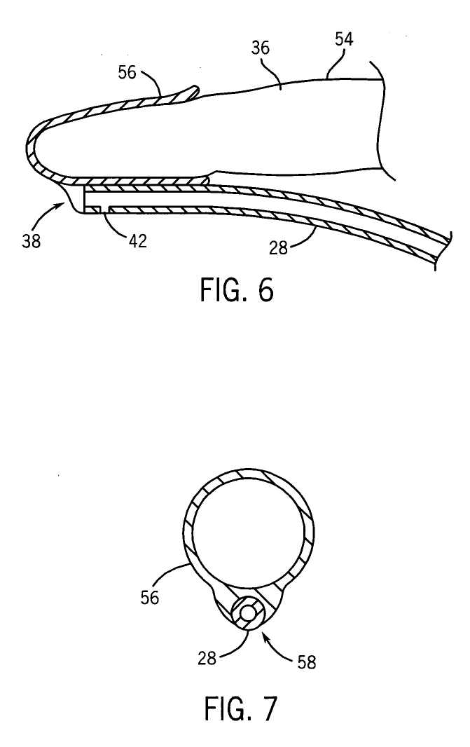

Referring now to FIGS. 6 and 7, in an alternative embodiment, the clamp 22 may be a finger cot 56 forming a cup fitting closely over the tip of the gloved finger 36. The finger cot 56 may be an elastomeric material to stretch slightly to be easily installed on and removed from the fingertip, but to resist accidental dislodgement once installed. The lower surface of the finger cot 56 may incorporate an axial channel 58 having sidewalls gripping the sides of the intake end 38 of the aspiration tube 28 so that the aspiration tube 28 may be snapped into engagement with the finger cot 56 with a variable or no cantilever and removed at will. Alternatively, the intake end 38 of the aspiration tube 28 may be permanently affixed to the finger cot 56 and the finger cot 56 removed when other use of the finger 36 is required.

It is specifically intended that the present invention not be limited to the embodiments and illustrations contained herein, but include modified forms of those embodiments including portions of the embodiments and combinations of elements of different embodiments as come within the scope of the following claims.

1. A hand mounted aspiration device for surgery comprising:
a flexible aspiration tube having an intake end receiving aspirated fluids; and
a clamp engaging a finger of a surgeon and holding the aspiration tube so that the intake end is grippable between a tip of the finger and a thumb.

2. The hand mounted aspiration device of claim 1 wherein:
the aspiration tube is axially bendable; and
wherein the clamp holds the aspiration tube at a portion removed from the intake end, to direct the intake end in cantilever fashion to a point grippable between a tip of the finger and a thumb.

3. The hand mounted aspiration device of claim 2 wherein the aspiration tube is elastic and the clamp holds the portion of the aspiration tube so that a grasping of the intake end between the tip of the finger and the thumb flexes the aspiration tube, and release of the intake end from gripping moves the intake end away from a gripping point.

4. The hand mounted aspiration device of claim 2 wherein the clamp is a ring encircling the finger.

5. The hand mounted aspiration device of claim 4 wherein the ring flexes to expand in diameter.

6. The hand mounted aspiration device of claim 2 wherein the clamp holds the aspiration tube to allow sliding adjustment of a length of cantilever of the aspiration tube from the clamp to the intake end.

7. The hand mounted aspiration device of claim 2 wherein the clamp holds the aspiration tube with a ring.

8. The hand mounted aspiration device of claim 7 wherein the ring allows sliding adjustment of the aspiration tube.

9. The hand mounted aspiration device of claim 2 wherein the clamp is a pair of co-planar rings.

10. The hand mounted aspiration device of claim 2 wherein the clamp is a single piece plastic element.

11. The hand mounted aspiration device of claim 2 further including a wristband stabilizing an end of the aspiration tube opposite the intake end against a wrist of the surgeon.

12. The hand mounted aspiration device of claim 2 wherein the aspiration tube terminates at an end opposite the intake end with a bayonet connector.

13. The hand mounted aspiration device of claim 2 wherein the intake end includes an axial opening and at least one radial opening.

14. The hand mounted aspiration device of claim 2 further including an aspiration flow valve for controlling the flow of aspirated materials.

15. A sterile kit for surgery comprising:
a sterile disposable pouch sealed over a hand mounted aspiration device for surgery including:
a sterile axially resilient aspiration tube having an intake end receiving aspirated fluids; and
a sterile clamp sized to engage a finger of a surgeon and attached to the aspiration tube at a portion removed from the intake end, to direct the intake end in cantilever fashion to a point grippable between a tip of the finger and a thumb of an average adult.
